(19) United States
(12) Patent Application Publication
(10) Pub. No.: US 2024/0206841 A1
(43) Pub. Date: Jun. 27, 2024

(54) Handheld Auscultation Systems, Devices, and Methods

(71) Applicant: Emory University, Atlanta, GA (US)

(72) Inventors: Matthew Timothy Brown, Atlanta, GA (US); Keval Bollavaram, Snellville, GA (US); Sil Savla, Johns Creek, GA (US); Atharv Vikram Marathe, Mabelton, GA (US); Abhiramgopal Akella, Lilburn, GA (US); Ahdil Gill, Roswell, GA (US)

(21) Appl. No.: 18/288,503

(22) PCT Filed: Apr. 26, 2022
(86) PCT No.: PCT/US2022/026327

§ 371 Date: Oct. 26, 2023

Related U.S. Application Data
(60) Provisional application No. 63/179,573, filed on Apr. 26, 2021.

(51) Int. Cl. A61B 7/04; A61B 5/00; A61B 5/282; A61B 5/308; A61B 5/33; A61B 5/332; A61B 5/333

(52) U.S. Cl. CPC A61B 7/04; A61B 5/0006; A61B 5/002; A61B 5/282; A61B 5/308; A61B 5/33; A61B 5/332; A61B 5/333; A61B 5/7225; A61B 5/742; A61B 2560/0431; A61B 2560/0462; A61B 2562/04; A61B 2562/06

(57) ABSTRACT

The disclosure relates to devices, systems and methods for cost effective and user-friendly handheld auscultation. In one implementation, the device may include a housing having a first end, a second end, and a circumference disposed there between. The device may further include a spherical bell disposed at the first end of the housing. The device may further include a microphone disposed below the spherical bell and configured to record auscultation signal data. The device may also include one or more cardiac sensors disposed along the circumference and configured to record cardiac signal data. The device may further include a microprocessor disposed in the housing and configured to process the auscultation and cardiac signal data.

### CROSS-REFERENCE TO RELATED APPLICATIONS

This application claims the benefit of U.S. Provisional Application No. 63/179,573 filed Apr. 26, 2021. The entirety of this application is hereby incorporated by reference for all purposes.

### BACKGROUND

In healthcare, telemedicine visits conducted over a variety of virtual platforms became much more commonplace. Despite the ease the connection of patients with providers, a clinician's ability to perform a thorough physical exam, such as listening to a patient's heart, lungs, and abdomen, can be limited during a telemedicine visit. This can hinder the physician's ability to provide thorough medical consultation to the patient. While there are several point of care devices available, they are not widely used because they can be costly and inaccurate, as well as not user friendly.

### SUMMARY

Thus, there is a need for accurate, cost-effective, and user-friendly device that can collect auscultation signal data.

Systems, devices, and methods disclosed herein relate generally to a hand-held user device that can simultaneously collect physiological data (e.g., auscultation signal data and/or cardiac signal data (e.g., electrocardiogram (ECG) data)). The systems, devices, and methods disclosed herein can be used by a patient, for example, during or in preparation of a remote, telemedicine visit, without physician oversight. The systems, devices, and methods disclosed herein can provide physicians with at least the auscultation signal data and cardiac signal data necessary to perform a health assessment of a patient, even when during a remote-consultation.

In some examples, the disclosed embodiments may include handheld auscultation devices. In some examples, a handheld auscultation device may include a housing having a first end, a second end, and a circumference disposed there between. The device may also include a spherical bell disposed at the first end of the housing. The device may also include a microphone disposed below the spherical bell and configured to record auscultation signal data. The device may further include one or more cardiac sensors disposed along the circumference and configured to record cardiac signal data. The device may also include a microprocessor disposed in the housing and configured to process the auscultation and cardiac signal data.

In some examples, the one or more cardiac sensors may include a first electrocardiogram sensor disposed at the second end of the housing so as to be disposed opposing the spherical bell. The first electrocardiogram sensor may be configured to be disposed against a palm of a user.

In some examples, the one or more cardiac sensors may include a second electrocardiogram sensor and a third electrocardiogram sensor. The second electrocardiogram sensor and the third electrocardiogram sensor may be disposed along the circumference of the housing. In some examples, the one or more cardiac sensors may be electrocardiogram sensor plate(s).

In some examples, the device may further include a strap coupled to the housing and disposed to loop around the second end of the housing. The strap may be configured to be looped around a hand of the user so that the first electrocardiogram sensor rests against the palm of the user.

In some examples, the one or more cardiac sensors and the microphone may be configured to simultaneously record the cardiac signal data and the auscultation signal data, respectively.

In some examples, the device may further include signal processing circuitry disposed within the housing.

In some examples, the bell may have a depth that is greater than 7.0 mm.

In some examples, the device may further include an interface configured for wired or wireless connection to a mobile device.

In some examples, the disclosed embodiments may include methods for processing signal data of a user (e.g., patient) using a handheld auscultation device. In some examples, a method may include simultaneously collecting auscultation signal data of the user using a bell and a microphone of the handheld auscultation device and cardiac signal data using one or more cardiac sensors of the handheld auscultation device at a contact point. In some examples, the method may further include processing, using the handheld auscultation device, the auscultation signal data and the cardiac signal data to generate digital auscultation signal data and digital cardiac signal data. The method may also include transmitting the digital auscultation signal data and the digital cardiac signal data to a mobile device (e.g., smart phone, tablet, etc.) connected to the handheld auscultation device.

In some examples, the one or more cardiac sensors may include a first electrocardiogram sensor disposed at the second end of the housing. The first electrocardiogram sensor may be configured to be disposed against a palm of the user.

In some examples, the one or more cardiac sensors may include a second electrocardiogram sensor and a third electrocardiogram sensor. The second electrocardiogram sensor and the third electrocardiogram sensor may be disposed along the circumference of the housing. In some examples, the one or more cardiac sensors may be electrocardiogram sensor plate(s).

In some examples, the device may further include a strap coupled to the housing and disposed to loop around the second end of the housing. The strap may be looped around a hand of the user so that the first electrocardiogram sensor rests against the palm of the user.

In some examples, the bell may have a depth that is greater than 7.0 mm.

In some examples, the transmission may be performed using wired or wireless connection.

In some examples, the method may further include receiving, at the mobile device, the digital auscultation signal data and the digital cardiac signal data for the contact point. The method may also include storing, at the mobile device, the digital auscultation signal data and the digital cardiac signal data for the contact point. In some examples, the digital auscultation signal data may be stored as an audio file and the digital cardiac signal data may be stored as a data file.

In some examples, the method may further include processing the digital auscultation signal data and the digital cardiac signal data to determine one or more clinical parameters.

In some examples, the method may also include generating a user interface using the digital auscultation signal data and the digital cardiac signal data. The user interface may include a visualization of waveforms associated with the digital auscultation signal data and the digital cardiac signal data.

In some examples, the processing, using the handheld auscultation device, the auscultation signal data and the cardiac signal data to generate the digital auscultation signal data and the digital cardiac signal data may include converting the auscultation signal data and the cardiac signal data to the digital auscultation signal data and the digital cardiac signal data. In some examples, this processing may further include filtering the digital auscultation signal data and the digital cardiac signal data to remove noise.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1A illustrates an example of system environment in which a handheld auscultation device may be implemented for example, for remote health monitoring. according to embodiments; and FIG. 1B shows an enlarged view of the user interface shown in FIG. 1A.

FIGS. 2A-D show views of the handheld auscultation device. FIG. 2A shows a perspective view of the handheld auscultation device; FIG. 2B shows a bottom view of the handheld auscultation device; FIG. 2C shows an exploded view of the handheld auscultation device; and FIG. 2D shows another exploded view of the handheld auscultation device.

FIG. 3 shows a table of a comparison of loudness and ratio of signal power to the noise power (SNR) of different bell shape devices.

FIG. 4 is block diagram of the general functionality of a signal processing circuitry of the handheld auscultation device.

FIG. 5 shows a flowchart illustrating an example of a method of processing auscultation signal data and cardiac signal data at the handheld auscultation device.

FIG. 6 shows a flowchart illustrating an example of a method of processing the auscultation signal data and the cardiac signal data to determine one or more clinical parameters.

FIG. 7 is a simplified block diagram of an example of a computing system for implementing certain embodiments disclosed herein.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments relate to a handheld auscultation device that can collect at least auscultation signal data. In some examples, the handheld device may be configured to collect auscultation signal data, cardiac signal data (e.g., ECG data), other physiological signal data, among others, or a combination thereof. This device can be cost-effective and simple to use for a layperson so that a user can easily and accurately collect auscultation signal data and/or cardiac signal data for personal use, as well as remotely, for telemedicine visits as well as for auscultation and/or physiological signal diagnostic applications. Additionally, it can be easily used by a physician to collect auscultation signal data and/or cardiac signal data of a patient at in-office visits.

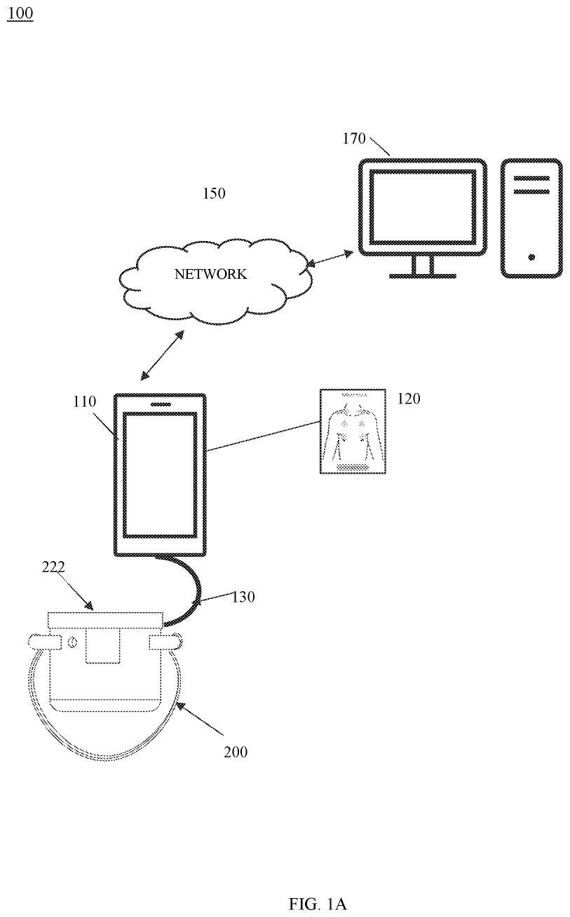

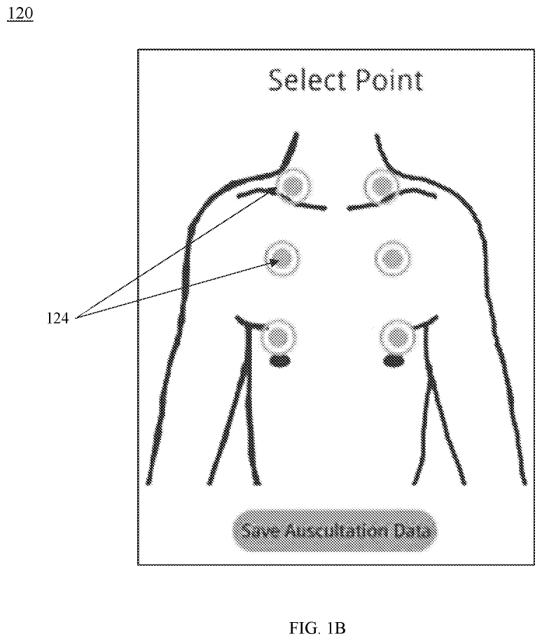

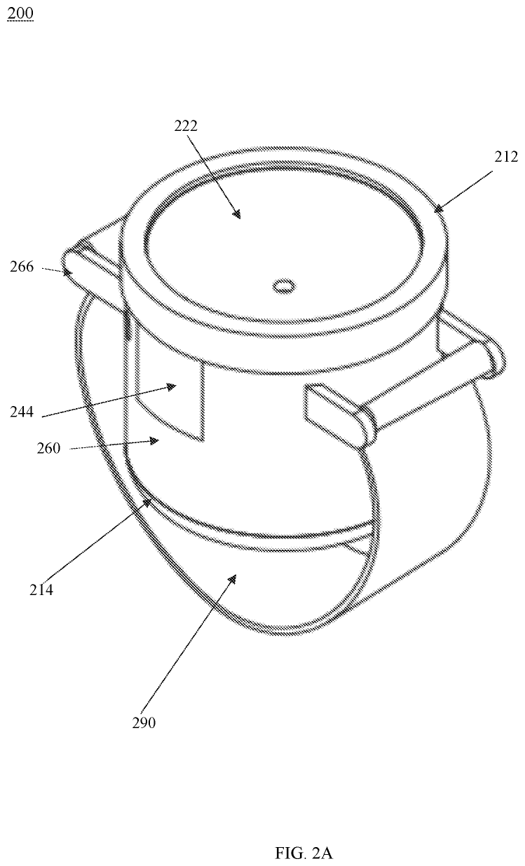

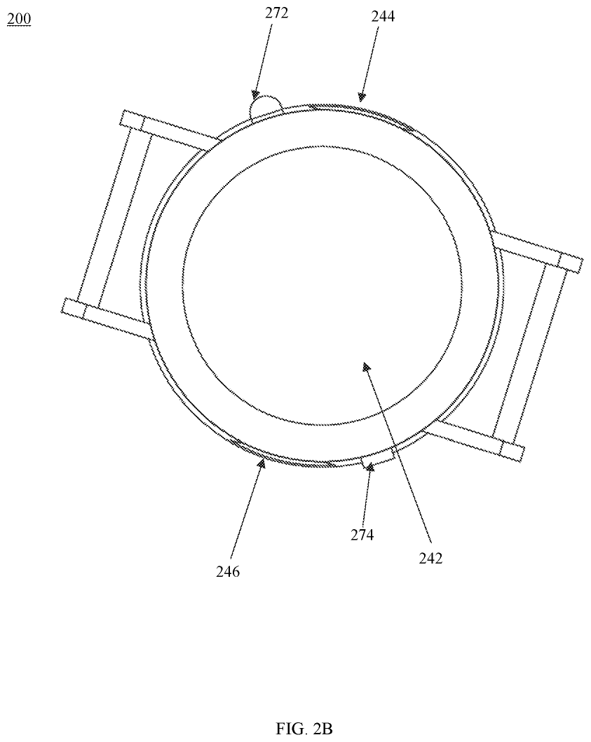

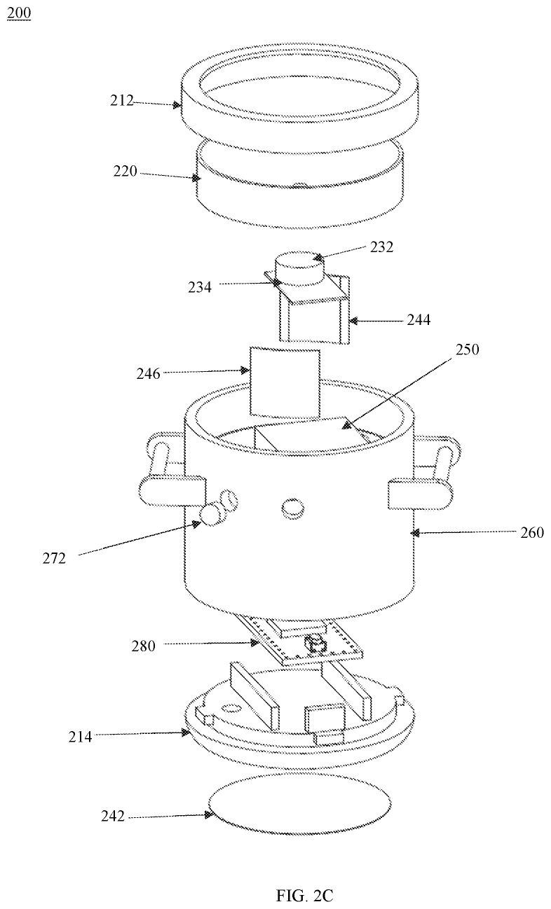

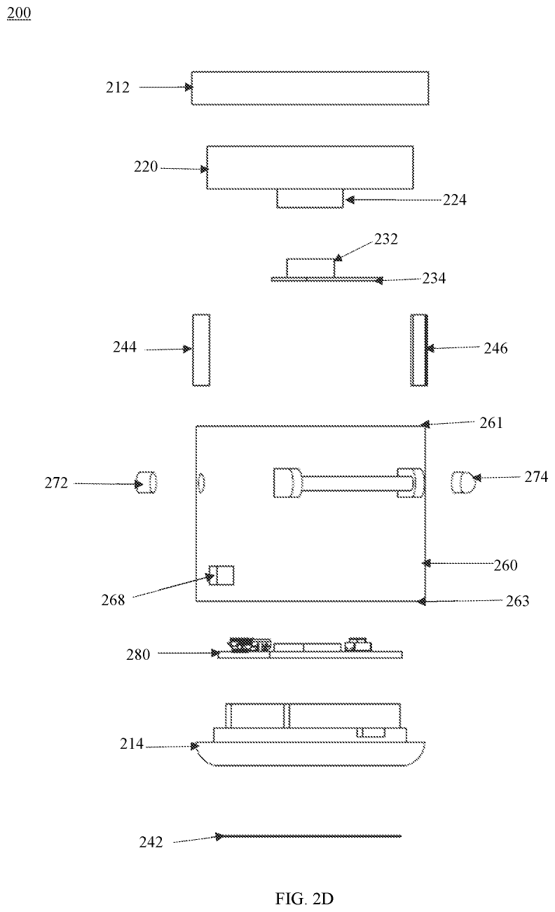

FIG. 1A depicts an example of a system environment 100 in which a handheld auscultation device 200 may be implemented for remote health monitoring. In some examples, the handheld auscultation device 200 may be used by a user (e.g., also referred to as “patient”) to record and process at least auscultation signal data at one or more contact points. For example, the device 200 may be configured to be disposed in a palm of a user so that a bell 222 is exposed so that it can be held against the body of the user at a contact point on their anatomy to record and process at least auscultation signal data. In some embodiments, the device 200 may configured to simultaneously record and process at least auscultation signal data and cardiac signal data at each target on the user's anatomy. FIGS. 2A-2D show additional views of the device 200.

In some examples, the device 200 may be configured to be connected to a user device 110 via a wired and/or wireless connection 130. The user device 110 may include any computing or data processing device consistent with the disclosed embodiments. In some embodiments, the device 110 may incorporate the functionalities associated with a personal computer, a laptop computer, a tablet computer, a notebook computer, a hand-held computer, a personal digital assistant, a mobile phone, an embedded device, a smartphone, and/or any additional or alternate computing device/system. In some examples, the device 200 may be connected to the device 110 using a cable, such as a 4-pole audio cable, micro-USB cable, USB cable, and/or Lightning cable, to transmit the collected auscultation (e.g., audio) signal data and cardiac signal data to the user device 110. In this example, the device 200 may include the corresponding interface, such as a port (e.g., 4-pole audio port, micro-USB port, USB port, Lightning port, etc.)(e.g., port 268 described below). In some examples, the device 200 may additionally and/or alternatively be configured to wirelessly transmit the signal data to the device 110, for example, using Wi-Fi®, a Zigbee®, a Bluetooth®, among others, or any combination thereof. In this example, the device 200 may additionally and/or alternatively include the corresponding communication interface, such as Wi-Fi® interface, a Zigbee® interface, a Bluetooth® interface, among others, or any combination thereof.

In some examples, the user device 110 may include an auscultation module (e.g., an application) that enables the user device 110 to communicate with the device 200 and provide instructions to guide a user through the placement and use of the device 200 (e.g., via a series of prompts on a user interface/display on the user device 110) at different contact points on the user's anatomy (e.g., chest, neck, stomach, back, etc.) according to selected mode. By way of example, the auscultation module may be configured for one or more modes. By way of example, the one or more modes may include but is not limited to cardiac, pulmonary, carotid artery, gastrointestinal, among others, or any combination thereof.

In some examples, the auscultation module may be configured to generate and display a user interface 120 showing contact points on the user's anatomy (e.g., chest, neck, stomach, back, etc.) to instruct a user to record auscultation signal data and cardiac signal data at the locations on their chest/black corresponding to the displayed contact points using the device 200. FIG. 1B shows an example of a generated user interface for the pulmonary mode showing a plurality of contact points 124 on the front/chest of a user. In this example, the user would collect data for each contact point. After the device 200 records and processes the data for each contact point, the device 200 may transmit the data to the user device 110 using a wired or wireless connection. In some examples, the device 200 may be configured to separately process the collected auscultation signal data and cardiac signal data, for example, to remove noise from the collected raw data for each target. The user device 110 may then locally store the processed data with respect to that contact point. The module may instruct the user to record auscultation signal and cardiac signal data for multiple contact points on the chest and sides of the body of the user.

The user device 110 (the module) may can be configured to communicate, for example, using a network 150, with a healthcare provider device and/or a remote server 170 operated by a healthcare provider. In some examples, (i) the user device 110 and/or (ii) the healthcare provider device and/or the remote server 170 may be configured to include a module (application) or be in communication with a module (application) on another server to further process the received auscultation signal data and/or cardiac signal data for one or more contact points to determine one or more clinical parameters. For example, the one or more clinical parameters may relate to cardiac parameters, respiratory parameters, gastrointestinal parameters, among others, or any combination thereof. By way of example, for the auscultation signal data, the device 110 and/or 170 may be configured to process the auscultation signal data for one or more contact points to determine heartbeats, pulse, respiratory rate, among other parameters, adventitious noises, among others, or any combination thereof. By way of another example, for the cardiac signal data, the device 110 and/or 170 may be configured to process the cardiac signal data for one or more contact points to determine heartbeats, pulse, heart rate variability measures, respiratory rate, abnormal heartbeats (e.g., arrhythmias), among others, or any combination thereof. In some examples, the one or more clinical parameters may be determined using the auscultation and/or cardiac signal data for each contact point.

In some examples, machine learning algorithms may be used to process the auscultation signal data and/or cardiac signal data for one or more points to extract clinically relevant features related to the one or more clinical parameters. For example, machine learning algorithms may be configured to process the cardiac signal data and/or auscultation signal data for one or more data points to detect and label normal and/or abnormal heartbeats.

In some examples, the module provided on the healthcare provider device and/or the remote server 110 and/or 170 may be configured to generate an interface in which a plot (e.g., waveform) of the auscultation signal data and/or cardiac signal data is displayed in time domain with zooming and sliding capabilities. For example, a frequency-domain representation may be generated and displayed with regards to each contact point (and/or more than contact point), displayed and compared against those from healthy reference data, the clinically relevant features/parameters, among others, or any combination thereof.

As described herein, appropriate programming (i.e., computer program, software, hardware, firmware, etc.) can be installed on or provided with each of each device 200, each user device 110, and/or the healthcare provider device and/or remote server 170, and can operate in a coordinated fashion to perform the methods of the present disclosure.

By way of example, the communication network 150 can include one or more networks such as a data network, a wireless network, a telephony network, or any combination thereof. The data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, NFC/RFID, RF memory tags, touch-distance radios, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Internet Protocol (IP) data casting, satellite, mobile ad-hoc network (MANET), and the like, or any combination thereof. The device 200, the device 110 and/or the device 170 may further implement aspects of the disclosed embodiments without accessing other devices or networks, such as network 150.

Although the systems/devices of the environment 100 are shown as being directly connected, the device 110 may be indirectly connected to one or more of the other systems/devices of the environment 100. In some embodiments, the device 110 may be only directly connected to one or more of the other systems/devices of the environment 100.

It is also to be understood that the environment 100 may omit any of the devices illustrated and/or may include additional systems and/or devices not shown. It is also to be understood that more than one device and/or system may be part of the environment 100 although one of each device and/or system is illustrated in the environment 100. It is further to be understood that each of the plurality of devices and/or systems may be different or may be the same. For example, one or more of the devices of the devices may be hosted at any of the other devices.

FIGS. 2A-D show views of the device 200. In some examples, the device 200 can include a bell 220 having an outer surface 222 that is configured to physically contact the body of the user at each contact point and to direct audio to a focal point in order to focus sound (without a diaphragm). In some examples, the bell 220 may be configured to receive and transmit sounds (without a diaphragm) from the chest, back, neck, stomach, among others, or a combination thereof of the user, for example, at position(s) corresponding to the contact point(s) (e.g., 120) displayed on the user device 110. By way of example, the bell may be configured to receive and transmit audio frequencies suited for audio tones used for heart sounds, lung sounds, gastrointestinal sounds, among others, or any combination thereof.

In some examples, the outer surface 222 of the bell 220 may have a spherical, concave shape. In some examples, the bell 220 may have a smooth inner surface. In some examples, the bell 220 may have a spherical, concave shape with a depth of about 6.9-7.5 mm.

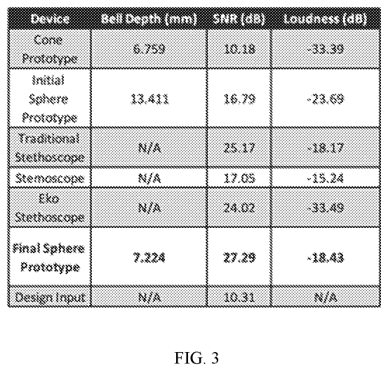

FIG. 3 show the results of testing of different bell shapes with respect to signal-to-noise ratio (SNR) and loudness. By way of example, the SNR was determined for e prototype devices and a few commercially available devices for comparison against a design input, which corresponds to an engineering design goal of an SNR of about 10.1 dB. The SNR for each device was determined by collecting an audio signal from an auscultation point (Papoint) and audio signal from a same (Pipoint) inert part of the body (e.g., a position with no internal sounds, such as bicep) with that device. In this table, SNR=10 log (Papoint1/Pipoint). As shown in FIG. 3, the final sphere prototype having a depth of 7.224 mm had the highest SNR of 27.39 dB. The higher SNR could enable accurate and efficient detection of pulmonary abnormalities from the recorded data.

By way of another example, loudness was determined for each device to ensure the prototypes captured audio that had sufficient audibility and loudness to distinguish sound for use by a physician, such as distinguishing different phases of the cardiac cycle. In this example, loudness was determined by measuring sound intensity for each device. Sound intensity of each device was measured by recording a signal from the same auscultation point and determining the maximum amplitude of the recorded waveform. As shown in the table in FIG. 3, the final sphere prototype having a depth of 7.2444 mm also had sufficient loudness.

In some examples, the device 200 may include a housing 260 having a first end 261, a second end 263, and a circumferential surface disposed thereof. In some examples, the bell 220 may be configured to be disposed on the first end 261 of the housing 260. In some examples, the bell 220 may be attached to the housing 260 using a cap 212 so that the conical/concave surface 222 of the bell 220 is exposed. In some examples, the device 200 may include a microphone (or transducer) 232 and a gain amplifier circuit 234 disposed between the bell 220 and circuit/power component(s) 250 disposed within the housing 260. In some examples, the microphone 232 may be disposed in a complimentary member 224 of the bell 220. For example, the member 224 may be an opening corresponding to a size of the microphone 232 so that the microphone 232 may be disposed within the member 224. In some examples, the microphone 232 may be configured to convert the acoustic/audio signal corresponding to the auscultation signal data into at least electrical signal data. The amplifier circuit 234 may be configured to amplify the electrical signal data corresponding to the auscultation signal data.

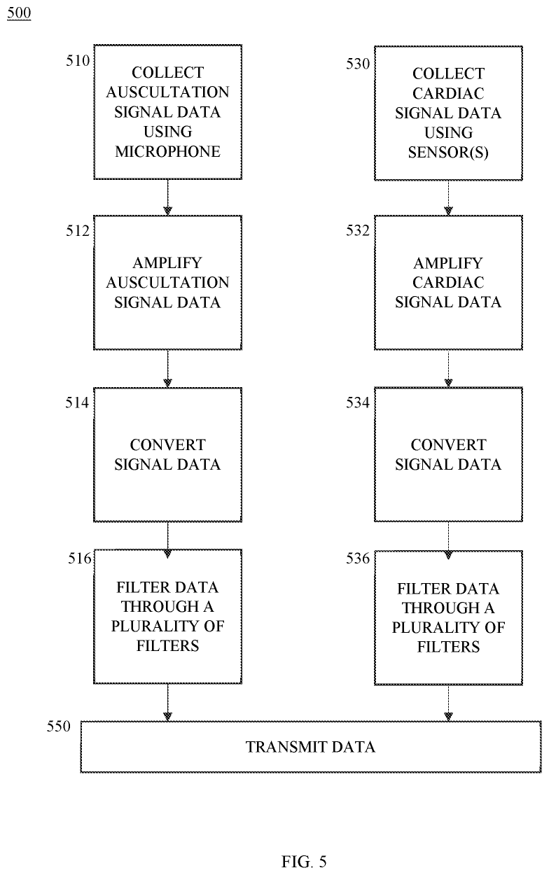

In some examples, the circuit/power component 250 may house one or more circuits and a power source. For example, the one or more circuits may include a filter circuit (e.g., low pass filter) for the auscultation signal data, a filter circuit for the ECG circuit (e.g., differential amplifier with a high and low pass filters), among others, or a combination thereof. FIG. 5 shows an example of the components of the circuit component(s) 250. In some examples, the power source may include but is not limited to battery, such as a rechargeable battery which may be recharged using a complimentary interface (e.g., port 268).

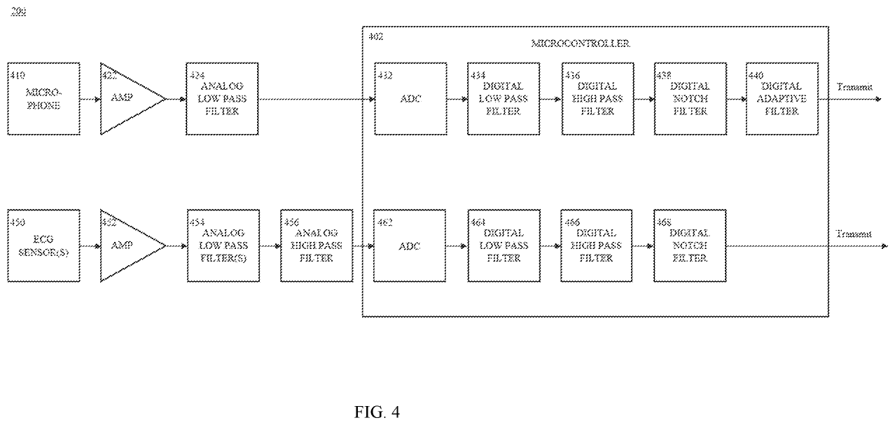

In some examples, the device 200 may also include a microcontroller 280 disposed within the housing. In some examples, the microcontroller 280 may be configured to process the auscultation signal data and cardiac signal data, for example, as described with respect to FIGS. 4 and/or 5.

In some examples, the device 200 may include a base member 214 disposed on the second end 263 of the housing 260.

In some examples, the device 200 may include one or more ECG sensors. The one or more ECG sensors is not limited to the ECG electrode plates (also referred to as ECG plates) as shown and may include alternative and/or additional ECG sensor(s). For example, the one or more ECG electrode plates may include but is not limited to ECG dry electrode, ECG foam electrode, other ECG electrode plates, other ECG sensors, among others, or any combination thereof. In this example, the one or more ECG sensors may include a first sensor (e.g., plate) 242, a second sensor (e.g., plate) 244, and a third sensor (e.g., plate) 246. By way of example, the first sensor 242 may be disposed at the second end 263 on an external surface of the base member 214. By way of another example, the second sensor 244 and the third sensor 246 may be disposed so as to be exposed along circumference of the housing 260 in opposing positions. In use, when holding the device 200 so that the first sensor 242 is against the user's palm and the surface 222 of the bell 220 is in contact with a position on the user's anatomy, such as the chest of the user, corresponding to the displayed/selected contact point, the user's fingers can also touch the sensors 244 and 246. This way, the device 200 can be configured to simultaneously record and process auscultation signal data and cardiac signal data at each contact point in which they are collected. In other examples, the device 200 may include more or less ECG sensors.

In some examples, the device 200 may include one or more tether points/clips 266 disposed on the housing 260 and configured to receive a strap 290. In this example, the device 200 may include two tether points/clips 266 disposed on opposite sides of the housing 260. For example, as shown in FIG. 2A, the strap 290 strap may be disposed on the tether points/clips 266 so as to loop around the base 214/ECG sensor 242. This way, the strap 290 can be configured to removably secure the device 200 to hand of the user so that the ECG sensor 242 is in contact with the palm of a user.

In some examples, the device 200 may further include a power button 272 and a LED 274 that can be configured to illuminate when the device 200 is powered on. In some examples, the device 200 may include a port 268 configured to charge the battery, connect the device 200 to a user device 110 (e.g., mobile device) using the corresponding cable, among others, or any combination thereof.

It will be understood that FIG. 2 may not illustrate all components of the device 200. The device 200 may include more or less components. For example, FIG. 2 may not illustrate all of the signal process circuitry components for processing the auscultation and/or cardiac signal data.

FIG. 4 shows an example of a simplified functional block diagram of the general functionality of signal processing circuitry disposed within the device 200 that may/may not be illustrated in FIG. 2.

As shown in FIG. 4, in some examples, the device 200 may include separate signal processing circuitry for the auscultation and cardiac signal data. In some examples, the device may include circuitry in connection with the microphone 410 (e.g., microphone 232) for processing the auscultation signal data and circuitry in connection with the ECG sensor(s) 450 (e.g., ECG sensors 242, 244 and 246) for processing the cardiac signal data.

In some examples, the microphone 410 may process the collected auscultation signal data (e.g., audio signals) to generate electric signal data. The microphone 410 may be in connection with a gain amplifier 422 that may be configured to be tunable by a potentiometer. The gain amplifier 422 may be in connection with one or more pass filters, such as an analog low pass filter 424 (e.g., 2000 Hz cutoff, 1× gain) that may process the amplified signal data to remove any signals above the threshold (2000 Hz cutoff, 1× gain). In some examples, the amplifier 422/analog low pass filter 424 may be integrated into a signal component (e.g., amplifier circuit 234). The amplifier 422/analog low pass filter 424 may be connected to the microcontroller 402 (e.g., microcontroller 280).

In some examples, the microcontroller 402 may include an analog-to-digital converter (ADC) 432, a digital low pass filter 434, a digital high pass filter 436, a digital notch filter 438, and an adaptive digital filter 440. By way of example, the ADC 432 may be configured to convert the (filtered) analog auscultation signal data from the analog low pass filter 424 to digital auscultation signal data. In some examples, the digital low pass filter 434 may be configured to have about 2000 Hz cutoff, the digital high pass filter 436 may be configured to have about 20 Hz cutoff, and the digital notch filter 438 may be configured to have a specified frequency range of about 60 Hz. In some examples, the adaptive digital filter 440 may be configured to minimize the error in the signal. By way of example, the adaptive digital filter 440 may use a least mean squares technique, for example, to modify the auscultation signal to best fit a smooth version. After the microcontroller 402 processes and filters the auscultation signal data, the microcontroller 402 may cause the (filtered) (digital) auscultation signal data to be sent to the user device 110.

In some examples, the ECG sensor(s) 450 may simultaneously detect cardiac signal data when recording auscultation signal data for a contact point using the microphone 410. The sensor(s) 450 may be in connection with a differential amplifier 452 that may be configured to have a 10× gain. The amplifier 452 may be in connection with one or more analog pass filters, such as an analog low pass filter 454 and an analog high pass filter 456, to process the amplified cardiac signal data. For example, the analog low pass filter may have a 100 Hz cutoff, 10× gain and the analog high pass 456 filter may have a 0.5 Hz cutoff, 10× gain. In some examples, the amplifier 452/pass filters 454 and 456 may be integrated into a signal component. The amplifier 452/pass filters 454 and 456 may be connected to the microcontroller 402 (e.g., microcontroller 280).

In some examples, the microcontroller 402 may include an ADC 462, a digital low pass filter 464, a digital high pass filter 466, and a digital notch filter 468 to process the cardiac signal data. By way of example, the ADC 462 may be configured to convert the (filtered) analog cardiac signal data from the filter 456 to digital cardiac signal data. In some examples, the digital low pass filter 464 may be configured to have about 100 Hz cutoff, the digital high pass filter 466 may be configured to have about 0.5 Hz cutoff, and the digital notch filter 468 may be configured to have a specified frequency range of about 60 Hz. After the microcontroller 402 processes and filters the cardiac signal data, the microcontroller 402 may cause the (filtered) (digital) cardiac signal data to be sent to the user device 110.

It will be understood that the device 200 may include/use additional and/or alterative signal processing circuitry, such as additional and/or alternative filters, amplifications, thresholds, frequency ranges, and/or filtering techniques.

FIG. 5 shows a flow chart 500 illustrating examples of a method of processing auscultation and cardiac signal data for each contact point according to certain embodiments. Operations described in flow chart 500 may be performed by the auscultation device, such as the device 200, described above with respect to FIGS. 1-4. Although the flow chart 500 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in flow chart 500 may begin at block 510, after a user pairs a mobile device having the auscultation module (e.g., application downloaded) with the auscultation device, the user places the device 200 on their hand so that the strap is looped around the user's hand and the sensor 242 is against the user's palm and the user's fingers are against the sensors 244 and 246, and the user selects a contact point on the user interface on the device 110 and places the device on a position on the user's body corresponding to that contact point, the device 200 may simultaneously collect auscultation signal data using the microphone 232/410 and cardiac signal data using the ECG sensor(s) for a period of time. Next, operations at blocks 512-516 to process the auscultation signal data and operations at blocks 532-536 to process the cardiac signal data may be performed in parallel.

At block 512, the device 200 may amplify the collected auscultation signal data, for example, using the amplifier 422/234. At block 512, the device 200 may filter the amplified auscultation signal data using a low pass filter, such as the analog low pass filter 424. In some examples, the device 200 may filter the amplified auscultation signal data using a 2000 Hz cutoff, 1× gain.

Next, at block 514, the device 200 may convert the filtered (analog) auscultation signal data to digital auscultation signal data, for example, using the ADC 432. Next, at block 516, the device 200 may filter the digital auscultation signal data through a plurality of filters. For example, the device 200 may filter the digital auscultation signal data through a digital low pass filter (e.g., digital low pass filter 434) with a 2000 Hz cutoff, through a digital high pass filter (e.g., digital high pass filter 438) with a 20 Hz cutoff, and a digital notch filter (e.g., digital notch filter 438) with a specified range of 60 Hz. In some examples, the device 200 may further filter the filtered digital auscultation signal through an adaptive filter (e.g., digital adaptive filter 440).

At block 532, the device may amplify the cardiac signal data, for example, using an amplifier such as the differential amplifier 452 having a 10× gain. At block 532, the device 200 may filter the amplified (analog) cardiac signal data using a plurality of pass filters, for example, the filters 454 and 456. By way of example, the device 200 may filter the amplified (analog) cardiac signal data through an analog lowpass filter (e.g., filter 454) having a 100 Hz cutoff, 10× gain, and an analog high pass filter (e.g., filter 456) having a 0.5 Hz cutoff, 10× gain.

Next, at block 534, the device 200 may convert the filtered (analog) cardiac signal data to digital cardiac signal data, for example, using the ADC 462. Next, at block 536, the device 200 may filter the digital cardiac signal data through a plurality of filters. By way of example, the device 200 may filter the digital cardiac signal data through one or more digital pass filters, such as a digital low pass filter (e.g., digital low pass filter 464) with a 100 Hz cutoff, a digital high pass filter (e.g., digital high pass filter 466) with an 0.5 Hz cutoff, and a digital notch filter (e.g., digital notch filter 468) with a specified range of 60 Hz.

After the device 200 processes the auscultation signal data (blocks 510-516) and the cardiac signal data (blocks 530-536), the device 200 may transmit, the processed (digital) auscultation signal data and cardiac signal data for that contact point to the user device 110 using wired and/or wireless interface(s)/connection(s).

It will be understood that the steps 510/530-550 may repeated for each contact point.

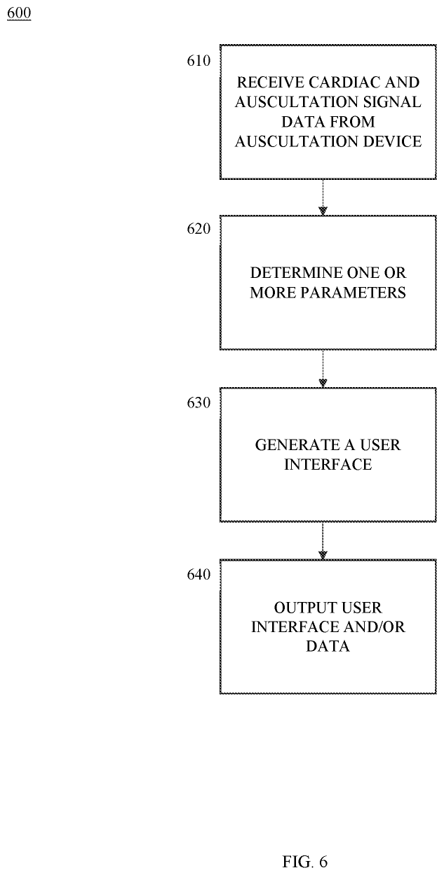

FIG. 6 shows a flow chart 600 illustrating examples of a method of processing auscultation and cardiac signal data for generating a user interface according to certain embodiments. Operations described in flow chart 600 may be performed by the devices 110 and/or 170, described above with respect to FIGS. 1A and B. Although the flow chart 600 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in flow chart 600 may begin at block 610, the device 110 receive may receive the processed (digital) auscultation signal data and cardiac signal data for a (selected) contact point from the auscultation device 200 (block 550). In some examples, the device 110 may store the auscultation and cardiac signal data locally in a directory for event usage as well as in a subdirectory in the associated contact point. For example, the auscultation signal data may be saved as an audio file, such as mp3, and the cardiac signal data may be saved as a data file. In some embodiments, the data file of the cardiac signal data may contain timestamps for each datapoint collected during the recording. In some examples, the device 110 may optionally transmit the processed (digital) auscultation signal data and cardiac signal data to the device 170 for storage (e.g., in the user's electronic health record (“EHR”), further processing (block 620), among others, or a combination thereof.

At block 620, the device 110 and/or 170 may process the stored (digital) auscultation and cardiac signal data to determine or more clinical parameters, for example, in response to a request for a display of the data. For example, the device 110 and/or 170 may process the data using feature detection algorithms to determine one or more clinical parameters including but not limited to pulse, heart variability measures, respiratory rate, etc.

Next, at block 630, the device 110 and/or 170 may generate a visualization of the (digital) auscultation and cardiac signal data (e.g., waveforms) and related parameters. In some examples, the device 110 and/or 170 may generate a user interface that can enable navigation of the visualization of the waveforms associated with the (digital) auscultation signal data and cardiac signal data, such as enabling the listening of the auscultation audio, navigation of the audio waveform using play, pause, forward, and rewind, etc.

At block 640, the device 110 and/or 170 may output the user interface, the auscultation and/or cardiac signal data, the parameter(s), among others, or a combination thereof. By way of example, the device 110 and/or 170 may display the user interface. In some examples, the device 110 may store the parameters, the user interface, and/or the data. In other examples, the device 110 may transmit the auscultation and/or cardiac signal data, the parameter(s), and/or user interface to the device 170 for display and/or storage (e.g., EHR of the user).

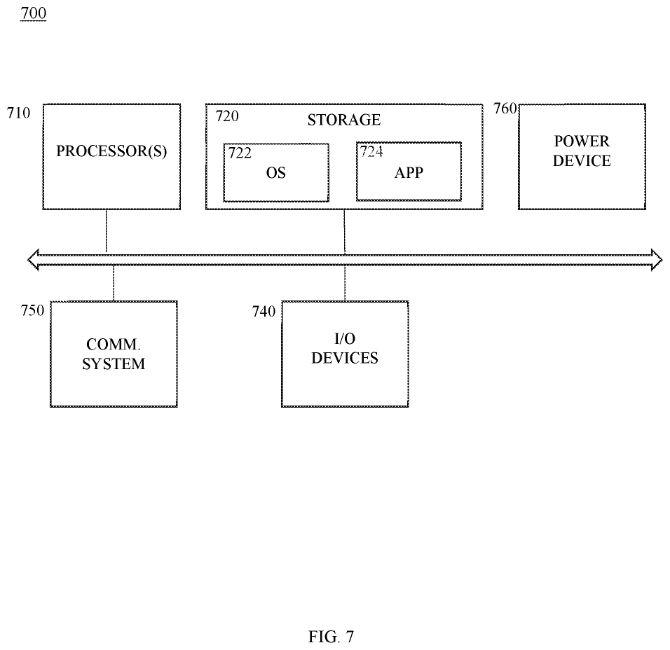

FIG. 7 depicts a block diagram of an example computing system 700 for implementing certain embodiments. For example, in some aspects, the computer system 700 may include computing systems associated with a device (e.g., the device 110 and/or 170) performing one or more processes (e.g., FIG. 6) disclosed herein. The block diagram illustrates some electronic components or subsystems of the computing system. The computing system 700 depicted in FIG. 7 is merely an example and is not intended to unduly limit the scope of inventive embodiments recited in the claims. One of ordinary skill in the art would recognize many possible variations, alternatives, and modifications. For example, in some implementations, the computing system 700 may have more or fewer subsystems than those shown in FIG. 7, may combine two or more subsystems, or may have a different configuration or arrangement of subsystems.

In the example shown in FIG. 7, the computing system 700 may include one or more processing units 710 and storage 720. The processing units 710 may be configured to execute instructions for performing various operations, and can include, for example, a micro-controller, a general-purpose processor, or a microprocessor suitable for implementation within a portable electronic device, such as a Raspberry Pi. The processing units 710 may be communicatively coupled with a plurality of components within the computing system 700. For example, the processing units 710 may communicate with other components across a bus. The bus may be any subsystem adapted to transfer data within the computing system 700. The bus may include a plurality of computer buses and additional circuitry to transfer data.

In some embodiments, the processing units 710 may be coupled to the storage 720. In some embodiments, the storage 720 may offer both short-term and long-term storage and may be divided into several units. The storage 720 may be volatile, such as static random-access memory (SRAM) and/or dynamic random-access memory (DRAM), and/or non-volatile, such as read-only memory (ROM), flash memory, and the like. Furthermore, the storage 720 may include removable storage devices, such as secure digital (SD) cards. The storage 720 may provide storage of computer readable instructions, data structures, program modules, audio recordings, image files, video recordings, and other data for the computing system 700. In some embodiments, the storage 720 may be distributed into different hardware modules. A set of instructions and/or code might be stored on the storage 720. The instructions might take the form of executable code that may be executable by the computing system 700, and/or might take the form of source and/or installable code, which, upon compilation and/or installation on the computing system 700 (e.g., using any of a variety of generally available compilers, installation programs, compression/decompression utilities, and the like), may take the form of executable code.

In some embodiments, the storage 720 may store a plurality of application modules 724, which may include any number of applications, such as applications for controlling input/output (I/O) devices 740 (a switch, a camera, a microphone or audio recorder, a speaker, a media player, a display device, etc.), interfacing with the devices 200 and/or 170, among others, or any combination thereof. The application modules 724 may include particular instructions to be executed by the processing units 710. In some embodiments, certain applications or parts of the application modules 724 may be executable by other hardware modules, such as a communication subsystem 750. In certain embodiments, the storage 720 may additionally include secure memory, which may include additional security controls to prevent copying or other unauthorized access to secure information.

In some embodiments, the storage 720 may include an operating system 722 loaded therein, such as an Android operating system or any other operating system suitable for mobile devices or portable devices. The operating system 722 may be operable to initiate the execution of the instructions provided by the application modules 724 and/or manage other hardware modules as well as interfaces with a communication subsystem 750 which may include one or more wireless or wired transceivers. The operating system 722 may be adapted to perform other operations across the components of the computing system 700 including threading, resource management, data storage control, and other similar functionality.

The communication subsystem 750 may include, for example, an infrared communication device, a wireless communication device and/or chipset (such as a Bluetooth® device, an IEEE 802.11 (Wi-Fi) device, a WiMax device, cellular communication facilities, and the like), NFC, ZigBee, and/or similar communication interfaces. The computing system 700 may include one or more antennas (not shown in FIG. 7) for wireless communication as part of the communication subsystem 750 or as a separate component coupled to any portion of the system.

Depending on desired functionality, the communication subsystem 750 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 802.9) network. A WLAN may be, for example, an IEEE 802.11x network. A WPAN may be, for example, a Bluetooth network, an IEEE 802.15x, or some other types of network. The techniques described herein may also be used for any combination of WWAN, WLAN, and/or WPAN. In some embodiments, the communications subsystem 750 may include wired communication devices, such as Universal Serial Bus (USB) devices, Universal Asynchronous Receiver/Transmitter (UART) devices, Ethernet devices, and the like. The communications subsystem 750 may permit data to be exchanged with a network, other computing systems, and/or any other devices described herein. The communication subsystem 750 may include a means for transmitting or receiving data, such as identifiers of portable goal tracking devices, position data, a geographic map, a heat map, photos, or videos, using antennas and wireless links. The communication subsystem 750, the processing units 710, and the storage 720 may together comprise at least a part of one or more of a means for performing some functions disclosed herein.

The computing system 700 may include one or more I/O devices 740, a switch, a camera, a microphone or audio recorder, a communication port, or the like. For example, the I/O devices 740 may include one or more touch sensors or button sensors associated with the buttons. The touch sensors or button sensors may include, for example, a mechanical switch or a capacitive sensor that can sense the touching or pressing of a button.

In some embodiments, the I/O devices 740 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP3, WAV, WMA, DSS, etc. The recorded audio files may be saved to the storage 720 or may be sent to the one or more network servers through the communication subsystem 750.

In some embodiments, the I/O devices 740 may include a location tracking device, such as a global positioning system (GPS) receiver. In some embodiments, the I/O devices 740 may include a wired communication port, such as a micro-USB, Lightning, or Thunderbolt transceiver.

The I/O devices 740 may also include, for example, a speaker, a media player, a display device, a communication port, or the like. For example, the I/O devices 740 may include a display device, such as an LED or LCD display and the corresponding driver circuit. The I/O devices 740 may include a text, audio, or video player that may display a text message, play an audio clip, or display a video clip.

The computing system 700 may include a power device 760, such as a rechargeable battery for providing electrical power to other circuits on the computing system 700. The rechargeable battery may include, for example, one or more alkaline batteries, lead-acid batteries, lithium-ion batteries, zinc-carbon batteries, and NiCd or NiMH batteries. The computing system 700 may also include a battery charger for charging the rechargeable battery. In some embodiments, the battery charger may include a wireless charging antenna that may support, for example, one of Qi, Power Matters Association (PMA), or Association for Wireless Power (A4WP) standard, and may operate at different frequencies. In some embodiments, the battery charger may include a hard-wired connector, such as, for example, a micro-USB or Lightning® connector, for charging the rechargeable battery using a hard-wired connection. The power device 760 may also include some power management integrated circuits, power regulators, power convertors, and the like.

The computing system 700 may be implemented in many different ways. In some embodiments, the different components of the computing system 700 described above may be integrated to a same printed circuit board. In some embodiments, the different components of the computing system 700 described above may be placed in different physical locations and interconnected by, for example, electrical wires. The computing system 700 may be implemented in various physical forms and may have various external appearances. The components of computing system 700 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The disclosures of each and every publication cited herein are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A handheld auscultation device, comprising:
a housing having a first end, a second end, and a circumference disposed there between;
a spherical bell disposed at the first end of the housing;
a microphone disposed below the spherical bell and configured to record auscultation signal data;
one or more cardiac sensors disposed along the circumference and configured to record cardiac signal data; and
a microprocessor disposed in the housing and configured to process the auscultation and cardiac signal data.

2. The device according to claim 1, wherein:
the one or more cardiac sensors includes a first electrocardiogram sensor disposed at the second end of the housing so as to be disposed opposing the spherical bell; and
the first electrocardiogram sensor is configured to be disposed against a palm of the user.

3. The device according to claim 2, wherein:
the one or more cardiac sensors includes a second electrocardiogram sensor and a third electrocardiogram sensor; and
the second electrocardiogram sensor and the third electrocardiogram sensor are disposed along the circumference of the housing.

4. The device according to claim 2, further comprising:
a strap coupled to the housing and disposed to loop around the second end of the housing.

5. The device according to claim 4, wherein the strap is configured to be looped around a hand of the user so that the first electrocardiogram sensor rests against the palm of the user.

6. The device according to claim 4, wherein the one or more cardiac sensors and the microphone are configured to simultaneously record the cardiac signal data and the auscultation signal data, respectively.

7. The device according to claim 2, further comprising:
signal processing circuitry disposed within the housing.

8. The device according to claim 1, wherein the bell has a depth that is greater than 7.0 mm.

9. The device according to claim 1, further comprising:
an interface configured for wired or wireless connection to a mobile device.

10. A method for processing signal data of a user using a handheld auscultation device, comprising:
simultaneously collecting auscultation signal data of a user using a bell and a microphone of the handheld auscultation device and cardiac signal data using one or more cardiac sensors of the handheld auscultation device at a contact point;
processing, using the handheld auscultation device, the auscultation signal data and the cardiac signal data to generate digital auscultation signal data and digital cardiac signal data; and
transmitting the digital auscultation signal data and the digital cardiac signal data to a mobile device connected to the handheld auscultation device.

11. The method according to claim 10, wherein:
the one or more cardiac sensors includes a first electrocardiogram sensor disposed at a second end of the housing; and
the first electrocardiogram sensor is disposed against a palm of the user.

12. The method according to claim 11, wherein:
the one or more cardiac sensors includes a second electrocardiogram sensor and a third electrocardiogram sensor; and
the second electrocardiogram sensor plate and the third electrocardiogram sensor are disposed along the circumference of the housing.

13. The method according to claim 11,
wherein the handheld auscultation device is strapped to a hand of the user so that the first electrocardiogram sensor rests against the palm of the user.

14. The method according to claim 10, wherein the bell has a depth that is greater than 7.0 mm.

15. The method according to claim 10, wherein the transmission is performed using wired or wireless connection.

16. The method according to claim 15, further comprising:
receiving, at the mobile device, the digital auscultation signal data and the digital cardiac signal data for the contact point; and
storing, at the mobile device, the digital auscultation signal data and the digital cardiac signal data for the contact point.

17. The method according to claim 16, wherein the digital auscultation signal data is stored as an audio file and the digital cardiac signal data is stored as a data file.

18. The method according to claim 17, further comprising:
processing the digital auscultation signal data and the digital cardiac signal data to determine one or more clinical parameters.

19. The method according to claim 18, further comprising:
generating a user interface using the digital auscultation signal data and the digital cardiac signal data, the user interface including a visualization of waveforms associated with the digital auscultation signal data and the digital cardiac signal data.

20. The method according to claim 10, wherein the processing, using the handheld auscultation device, the auscultation signal data and the cardiac signal data to generate the digital auscultation signal data and the digital cardiac signal data includes:
converting the auscultation signal data and the cardiac signal data to the digital auscultation signal data and the digital cardiac signal data; and
filtering the digital auscultation signal data and the digital cardiac signal data to remove noise.
